(19) United States
(12) Patent Application Publication
(10) Pub. No.: US 2001/0049090 A1
(43) Pub. Date: Dec. 6, 2001

(54) ENCODING OF ORGANOMETALLIC LIBRARIES

(76) Inventors: THOMAS BOUSSIE, MENLO PARK, CA (US); VINCE MURPHY, CUPERTINO, CA (US); JOHANNES A M VAN BEEK, MOUNTAIN VIEW, CA (US); MARTIN DEVENNEY, MOUNTAIN VIEW, CA (US); HOWARD W TURNER, CAMPBELL, CA (US); TIMOTHY POWERS, SAN FRANCISCO, CA (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/220,101

(22) Filed: Dec. 23, 1998

Related U.S. Application Data
(62) Division of application No. 09/033,207, filed on Mar. 2, 1998.

(51) Int. Cl. C12Q 1/00; G01N 31/10; B01J 31/00; B01J 27/26

(52) U.S. Cl. 435/4; 502/102; 502/400; 502/150; 502/104; 502/175; 436/34; 436/37

(57) ABSTRACT

The present invention discloses methods and materials for constructing combinatorial libraries composed of organometallic compounds immobilized on solid supports and encoded with detectable tags. The encoded library of organometallic compounds is especially useful for rapidly screening large numbers of member compounds for catalytic performance. These immobilized catalysts can be pooled in a single reactor where they are screened for a predefined property, such as catalytic activity and selectivity.

This application is a continuation-in-part of commonly assigned U.S. Provisional Application No. 60/056,818, filed Aug. 22, 1997, the complete disclosure of which is incorporated herein by reference.

This application is related to commonly assigned U.S. application Ser. No. 08/898,715, filed Jul. 22, 1997.

### BACKGROUND OF THE INVENTION

1. Technical Field

The present invention relates to methods and materials for screening large numbers of organometallic compounds for, among other properties, catalytic activity, and relates more particularly to methods and materials for constructing combinatorial libraries composed of immobilized organometallic compounds encoded with detectable tags.

2. Discussion

Organometallic compounds—substances bearing carbon-metal bonds—have long been used to catalyze commercially important chemical reactions. For example, lithium alkyls have been used with dienes to produce homopolymers of isoprene, butadiene, styrene and methyl methacrylate; Ziegler-Natta catalysts—mixtures of aluminum trialkyls and transition-metal chlorides—have been used to produce high-molecular weight, linear polyethylene and isotactic polypropylene.

Despite these successes, researchers' efforts to develop new organometallic catalysts have been hampered by the myriad factors affecting their performance. Speed and selectivity of organometallic catalysts may depend, for example, on reaction conditions used to prepare the catalyst, structure of the organic moiety, choice of metal, use of activators, and composition of solid supports used to immobilize the catalyst. Development efforts have been further hindered by the lack of a theoretical model that can accurately predict catalyst performance based on these factors.

Thus, though researchers have discovered a variety of organometallic catalysts over the past 15 years, progress has been slow. Traditionally, new catalysts have been developed by synthesizing relatively large amounts of candidate compounds and successively testing each compound for catalytic activity. Given the large number of factors affecting the performance of organometallic catalysts, the lack of a useful theoretical model, and the increasing demand for improved catalysts, researchers are continually seeking new approaches to the development of organometallic catalysts.

Significant progress has recently been achieved by abandoning the traditional development method in favor of a more rapid (e.g., parallel) approach to organometallic catalyst design. The approach relies on combinatorial synthesis and screening of a large library of organometallic compounds. The details of the approach are described in copending U.S. patent application Ser. No. 08/898,715 “Combinatorial Synthesis and Analysis of Organometallic Compounds and Homogeneous Catalysts” filed Jul. 22, 1997 (Docket No. 16703-000350), the teachings of which are incorporated herein by reference.

The use of solid phase supports for combinatorial organic synthesis is an important tool in the automated synthesis of dense libraries of organic compounds. Solid phase synthesis offers numerous advantages over conventional solution phase approaches. These advantages include ease of product isolation, the ability to use large excesses of reagents to drive slow reactions to completion, and a reduced tendency for yield limiting side reactions. Unfortunately, solid phase synthesis also suffers from a number of disadvantages including difficulties associated with fully characterizing solid phase reaction products.

One approach to constructing combinatorial libraries on a solid support is the split synthesis method. In this approach, the solid phase starting reagent is split into a number of equal portions that are separately treated with different reagents. The products are then pooled, mixed, and split once again into equal portions. Each portion is then treated with a second set of different reagents. The “split and pool” technique can be repeated to prepare dense libraries of organic compounds attached to a solid support. Resultant compounds may then be cleaved from the solid support to generate a soluble library of organic molecules that can be screened for a particular property. Currently, the pharmaceutical industry uses this technique for drug discovery.

Several encoding strategies have recently been described to aid in the deconvolution or identification of individual compounds that comprise the soluble library of organic molecules produced from split synthesis techniques. In these approaches, each set of reagents at every step of the synthesis is encoded (tagged) by separate molecules attached either to the compound itself or to the solid support. At the end of the solid phase split synthesis, each individual solid support material contains an organic molecule together with a unique set of tags that reveal its synthetic history. Encoding techniques include the use of oligonucleotide tags and peptide tags. See, for example, U.S. Pat. Nos. 5,639,603; 5,565,324; Maclean et al., 94 Proc. Nat'l Acad. Sci. USA 2805 (1997); Henderson et al., 117 J. Am. Chem. Soc. 5588 (1995). Other references will be known to those skilled in the art.

The use of encoding methodologies for the combinatorial synthesis and high throughput screening of organometallic compounds and catalysts would greatly enhance the efficiency of library preparation and subsequent screening. Pooling a mixture of different solid supported (immobilized) organometallic catalysts, suitably encoded so that individual compounds or solid supports could be accurately identified, would be a significant technological advance. Combinatorial synthesis, coupled with encoding of member compounds and solid supports, would allow screening of large numbers of immobilized organometallic catalysts in a single reactor, greatly improving the efficiency of catalyst development over traditional methods.

### SUMMARY OF THE INVENTION

In accordance with one aspect of the present invention, there is provided a method of identifying member compounds of a combinatorial library composed of organometallic compounds and/or inorganic complexes. The member compounds are immobilized on separate solid supports and are encoded by attaching one or more unique and detectable tags to each immobilized compound. The encoded member compounds are pooled and screened for a given property, and the tags are used to identify member compounds meeting a predefined screening criterion.

In accordance with a second aspect of the present invention, there is provided a method of screening member compounds of a combinatorial library for catalytic activity. The combinatorial library is composed of organometallic compounds and/or inorganic complexes. The member compounds are immobilized on separate solid supports and are encoded by attaching one or more unique and detectable tags to each immobilized compound. The encoded member compounds are combined and contacted with a reactant, and the tags are used to identify member compounds meeting a predefined measure of catalytic performance.

In accordance with a third aspect of the present invention, there is provided an encoded combinatorial library composed of solid supported (immobilized) organometallic and/or inorganic complexes. The member compounds have been encoded with one or more detectable and unique tags so that when the immobilized member compounds are combined with a reactant, each member compound exhibiting a predefined measure of catalytic performance can be identified by detecting its unique tag.

### BRIEF DESCRIPTION OF THE DRAWINGS

The foregoing and other advantages of the invention will become apparent upon reading the following detailed description and upon reference to the drawings in which:

FIG. 1 illustrates a synthetic scheme in which an olefin polymerization catalyst is immobilized on an encoded (tagged) support, is activated, and is exposed to an olefin monomer to produce a polymeric material that is embedded on the encoded solid support. Thereafter, the tags can be detected directly or removed chemically and subsequently detected to identify catalysts having desired properties.

FIG. 2 illustrates a synthetic scheme that can be used to attach secondary amine tags to a silica support.

FIG. 3 illustrates a synthetic scheme that can be used to achieve binary or higher encoding.

FIG. 4 illustrates a synthetic scheme that can be used to covalently attach a secondary amine tag to a silica support through the use of a trityl linker.

FIG. 5 illustrates a decoding scheme that can be used to decode secondary amine tags attached to silica supports.

FIG. 6 illustrates a synthetic scheme that can be used to attach a secondary amine tag to a functionalized polystyrene support.

FIG. 7 illustrates a synthetic scheme in which unreacted bromomethyl sites on a functionalized polystyrene support can be used, for example, to attach an olefin polymerization catalyst.

FIG. 8 illustrates a decoding scheme that can be used to decode secondary amine tags attached to functionalized polystyrene supports.

FIG. 9 illustrates a scheme that can be used to attach a fluorescent tag to a solid support.

FIG. 10 illustrates a scheme whereby a solid support encoded with fluorescent molecules can be decoded by measuring the convolved emission spectra at several excitation frequency wavelengths, followed by spectral deconvolution to identify the component fluorescent molecules.

FIG. 11 illustrates a synthetic scheme that can be used to attach fluorescent molecules to a silica support, followed by attachment of a catalyst to the support and polymerization of an olefin by the catalyst.

FIG. 12 illustrates a synthetic scheme in which the glass surface of a radiofrequency tag can be modified to allow organometallic compounds to be covalently attached to the tag.

FIG. 13 illustrates a synthetic scheme that can be used to functionalize a glass surface of a radiofrequency tag.

FIG. 14 illustrates a synthetic scheme in which the functionalized glass surface of a radiofrequency tag can be used to prepare encoded bis-imine ligands.

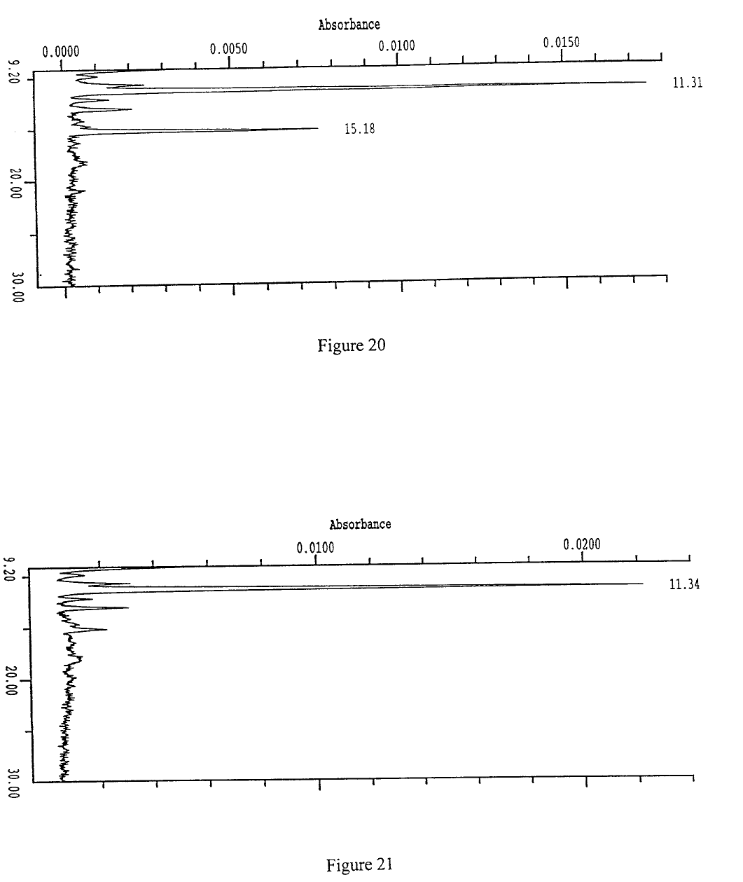

FIG. 15 provides an enlarged view of a representative sample of product that can be obtained from contacting a mixed pool of two tagged organometallic compounds with ethylene.

FIGS. 16-24 illustrate by example, and without limitation, HPLC traces that can be obtained from Samples 1-6 of Example 3.1, and from the sample described in Example 4.3, respectively.

FIG. 25 presents emission spectra obtainable from pyrene tagged silica before and after treatment with methylalumoxane.

FIG. 26 presents emission spectra obtainable from anthracene tagged silica before and after treatment with methylalumoxane.

### DESCRIPTION OF THE PREFERRED EMBODIMENTS

Overview

Generally, the encoding strategies of the present invention can be used to tag member compounds of combinatorial libraries composed of solid supported (immobilized) organometallic compounds as disclosed in copending U.S. Patent Application “Combinatorial Synthesis and Analysis of Organometallic Compounds and Homogeneous Catalysts,” filed Jul. 22, 1997 (Docket No. 16703-000350).

One embodiment provides a method of identifying member compounds of a combinatorial library. The combinatorial library is composed of N distinct organometallic compounds and/or inorganic complexes. Each member compound is immobilized on separate solid supports. Although the solid supports may each have the same composition, in general, they will be composed of M distinct compositions. Thus, each immobilized member compound can be represented by the formula AᵢBⱼ, where A represents the solid supports and B represents the member compounds; subscript i refers to a particular solid support composition and is any positive integer less than or equal to M, and subscript j refers to a particular member compound and is any positive integer less than or equal to N. Note that for each AᵢBⱼ, more than one copy of a particular member compound may be immobilized on the solid support.

The immobilized member compounds, AᵢBⱼ, are encoded by attaching at least one tag to each AᵢBⱼ so as to form tagged immobilized member compounds. Each tag (or group of tags) is detectable and is unique for each AᵢBⱼ. Generally, more than one copy of each AᵢBⱼ is tagged, e.g., more than one molecule AᵢBⱼ is tagged. The encoded member compounds are pooled and screened for a given property, and the tags are used to identify (decode) member compounds and solid supports meeting a predefined screening criterion, such as catalytic activity. The encoding and decoding methodologies are discussed in more detail below.

Encoding and Decoding

Tags may be attached covalently or adsorbed onto a solid support. An organometallic compound such as an olefin polymerization catalyst can then be adsorbed, covalently attached, or synthesized on the tagged solid support. Tags can also be attached directly to the organometallic compound, or upon screening for catalytic activity, may be attached to a reaction product.

Multiple tags can be attached to effect binary or higher encoding. Additionally, varying concentrations of one or more tags may be added and subsequently decoded quantitatively. Thus, varying concentrations of one or more tags can differentiate member compounds. When the organometallic compounds are synthesized on the solid supports, tags can be attached at every step of a “split and pool” synthesis, or they can be attached to the solid supports and used during the synthesis of a library using parallel methods. The tags can be read directly or, alternatively, can be chemically removed and subsequently read to identify, for example, the most active or the most selective catalysts or to identify the best solid support material.

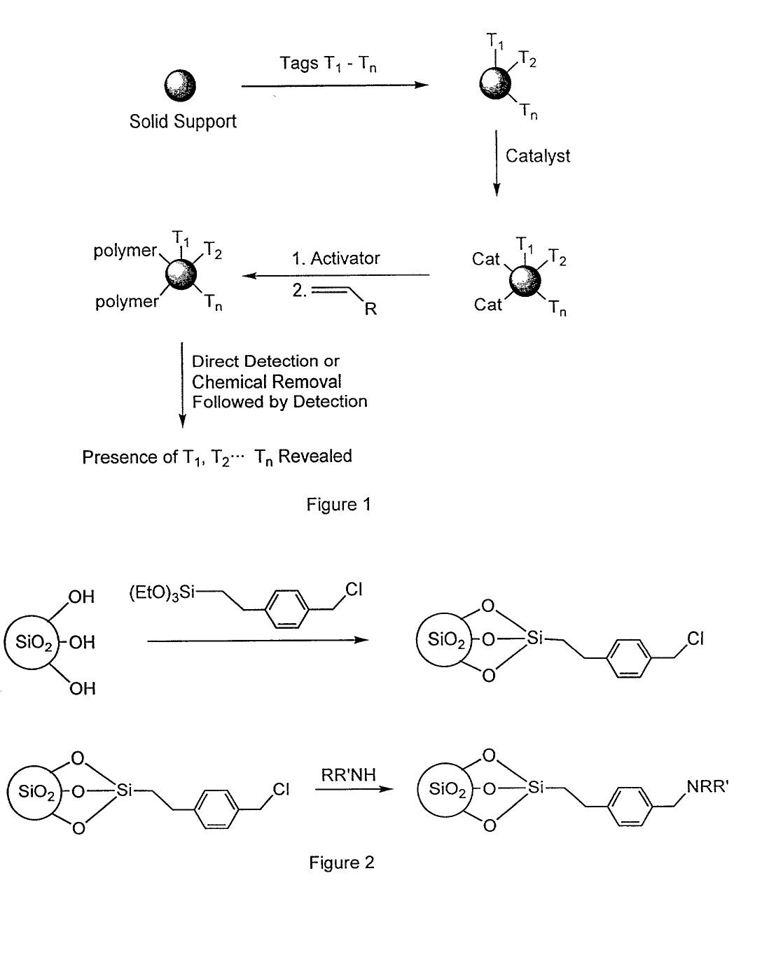

Of particular interest is the catalysis of polyolefins using organometallic compounds. In the case of an olefin polymerization catalyst immobilized on an encoded solid support, activation and exposure to an olefin monomer results in reaction products, C_{k,l} that adhere to the solid supported catalyst. Here, C represents the reaction products, subscript k is any positive integer less than or equal to M, and subscript l is any positive integer less than or equal to N. Each polyolefin molecule will adhere to the particular AᵢBⱼ that catalyzed its formation so that each C_{k,l} represents a particular reaction product catalyzed by AᵢBⱼ when k equals i and l equals j. The tags can be detected directly or, alternatively, chemically removed and subsequently detected to identify the solid support material or catalyst exhibiting the greatest activity or selectivity, or to identify immobilized organometallics that produce polymers having a desired morphology. This process is illustrated in FIG. 1.

Additionally, different solid materials can be tagged and screened for suitability as catalyst supports. Properties can vary within a class of solid supports, which can strongly influence catalyst performance. For instance, silica is an important solid support in the olefin polymerization industry, and it is well known that different silica samples can vary in surface area, pore volume, mean pore diameter, hydroxyl loading capacity, particle size, etc. These factors affect catalyst performance as measured by catalytic activity, comonomer incorporation, and polymer morphology. Thus, encoding different silica supports can substantially reduce the costs of optimizing a given catalyst system.

Tags

Any molecule, atom or ion, preferably a chemical, can be a useful tag in this invention, provided that the tag can be detected after screening the library. Chemical or readable tags suitable for use include, but are not limited to, amines and halocarbons. Chemical tags can be read or decoded by a variety of analytical techniques known by those of skill in the art. Such techniques include, for example, spectroscopy, High Performance Liquid Chromatography (HPLC) with fluorescence detection, Gas Chromatography (GC)-Mass Spectrometry (MS), Liquid Chromatography (LC)-MS, GC with Electron Capture Detection (GC-ECD). Fluorescent tags, and radiofrequency tags can also be used. Encoding can also be accomplished by nuclear transmutation and nuclear excitation.

Solid Supports

Solid support materials suitable for use include organic polymers, such as, but not limited to polystyrene, and inorganic solids, such as but not limited to silica. Solid supports may be selected from the group consisting of polystyrene, polysiloxane, polyethylene glycol, polypropylene glycol, polytetrafluoroethyelene, silica, alumina, aluminosilicate, magnesium chloride, and mixtures thereof.

1. Chemical Tagging of Silica

1.1 Covalent Attachment of Chemical Tag

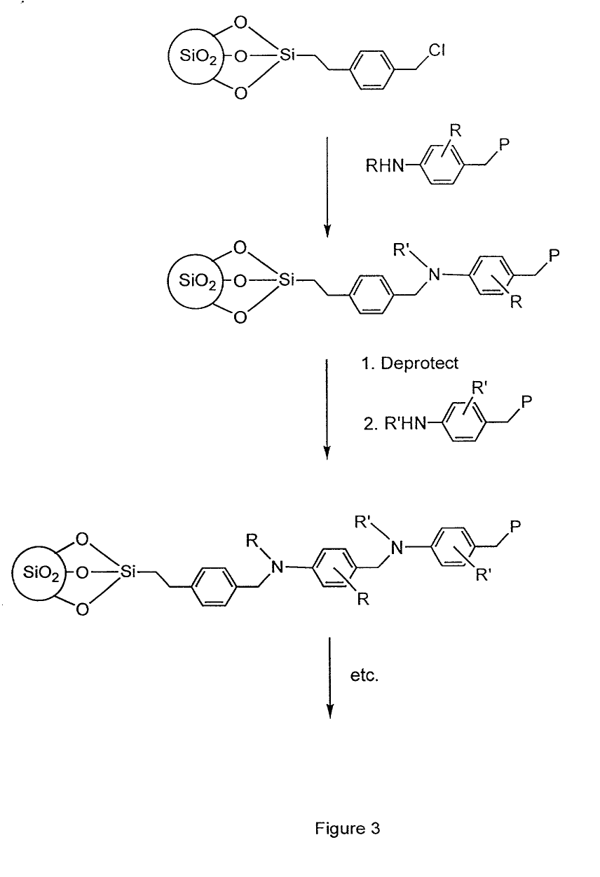

Silica can be encoded and decoded in various ways. For example, secondary amine tags can be attached to a silica support through the use of a silane coupling agent that can react with both the solid support and the amine. Referring to FIG. 2, the silane can be reacted with the silica support, followed by reaction with the amine. Alternatively, the amine can be first reacted with the silane, followed by reaction with the silica support. Other chemical or readable tags, such as halocarbons, can be similarly covalently linked to the silica support.

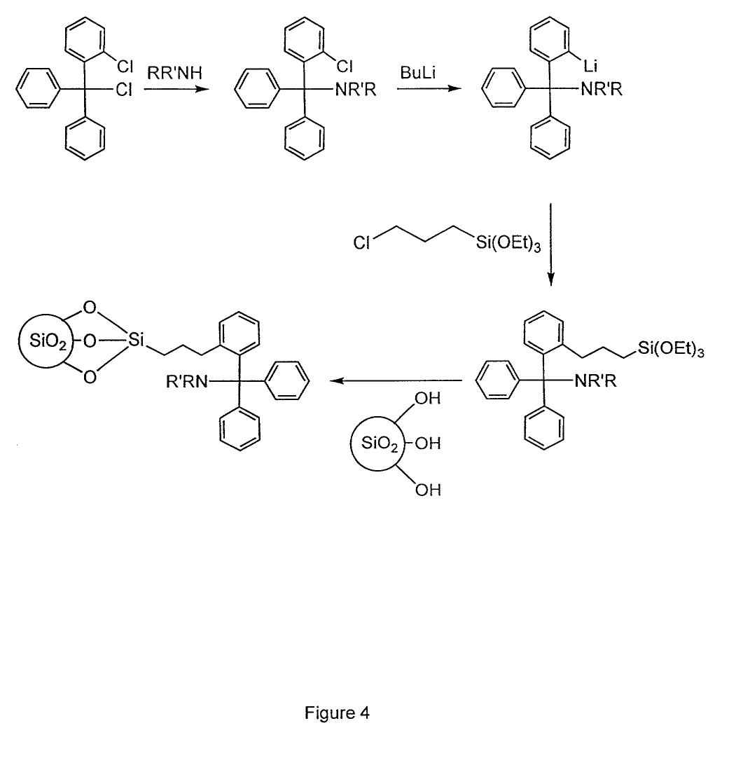

The secondary amine can also be attached to the silica support through the use of a trityl linker as illustrated in FIG. 3.

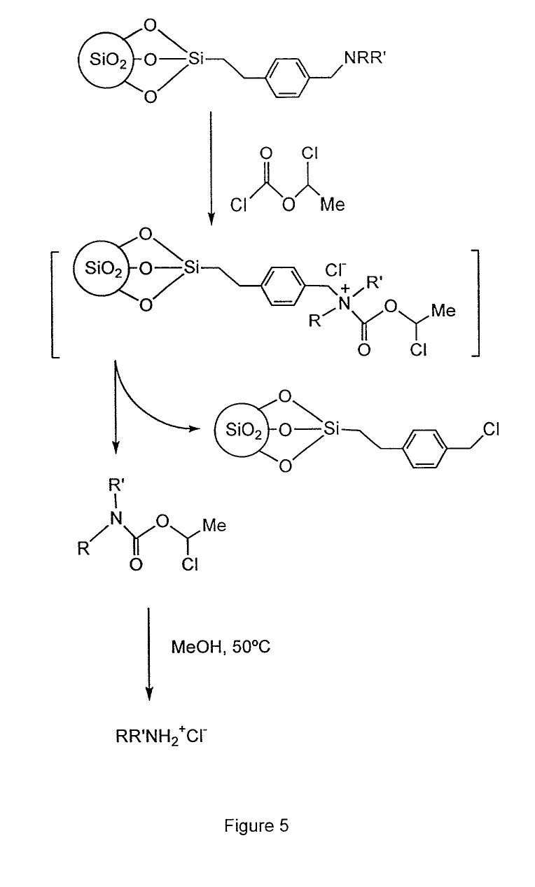

Multiple chemical tags can be covalently attached to the silica support to effect binary or higher encoding. As illustrated in FIG. 4, binary or higher encoding can also be accomplished by covalently linking tags to one another. Note that in FIG. 3, “P” represents an organic protecting group that is described, for example, in T. Greene, Protective Groups in Organic Synthesis (2d ed. 1981). Each amine can be attached at each stage of a split synthesis of an organometallic library.

Alternatively, covalent attachment can be accomplished through a variety of linkages including, but not limited to ester, amide, amine, ether, urea, thiourea, sulfonamide, alkyl, aryl and other linkages known to those skilled in the art. Organometallic compounds can then be adsorbed or covalently attached or synthesized on the encoded silica support and screened for a particular property such as the ability to catalyze olefin polymerization. Catalysts can be pooled in a single reactor, and screened for polymerization activity and product selectivity. Polymer products can then be decoded to reveal the identity of the catalyst. Alternatively, different silica supports can be tagged and screened for suitability as supports for olefin polymerization catalysts.

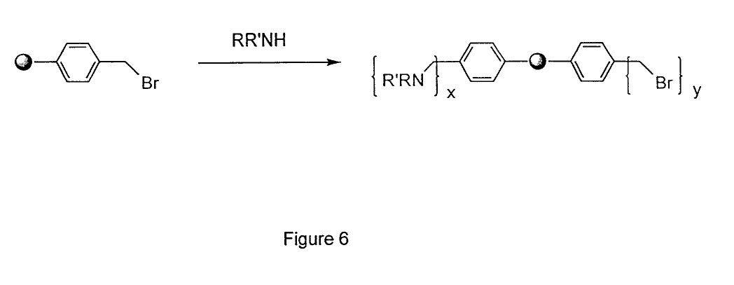

Decoding secondary amine tags from silica supports can be accomplished through the reaction sequence illustrated in FIG. 5. The hydrochloride salts of the secondary amines produced from the cleavage routine can be neutralized using a base, such as Li₂CO₃, derivatized with a fluorescent reagent, such as dansyl chloride, separated and then detected using HPLC coupled with fluorescence detection. Alternatively, the secondary amines can be separated and detected using LC-MS.

1.2 Adsorption of Chemical Tag

Chemical tags can be adsorbed onto silica supports. Multiple chemical tags can be attached to effect binary or higher encoding. Suitable tags include, but are not limited to, amines and halocarbons. After adsorption of the tags, the organometallic compounds can be adsorbed, covalently attached, or synthesized on the tagged silica supports, and then pooled and screened for a particular property, such as a catalytic activity. A particular organometallic compound and solid support can be decoded by first removing the chemical tags through, for example, acid washing. The chemical tags can then be separated and detected by various techniques. For instance, adsorbed amine tags can be treated with a fluorescent complexing agent, such as dansyl chloride, then separated by HPLC and identified by fluorescence detection. Alternatively, LC-MS can be used for separation and detection of amine tags. When adsorbed halocarbon tags are used, they can be separated and detected using techniques such as (without limitation) GC-ECD, GC-MS, or LC-MS.

2. Chemical Tagging of Functionalized Polystyrene

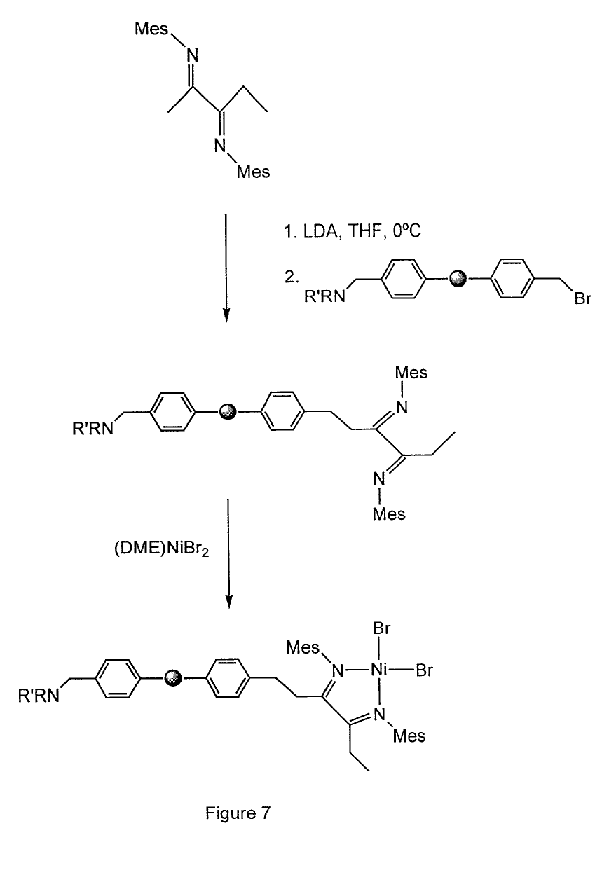

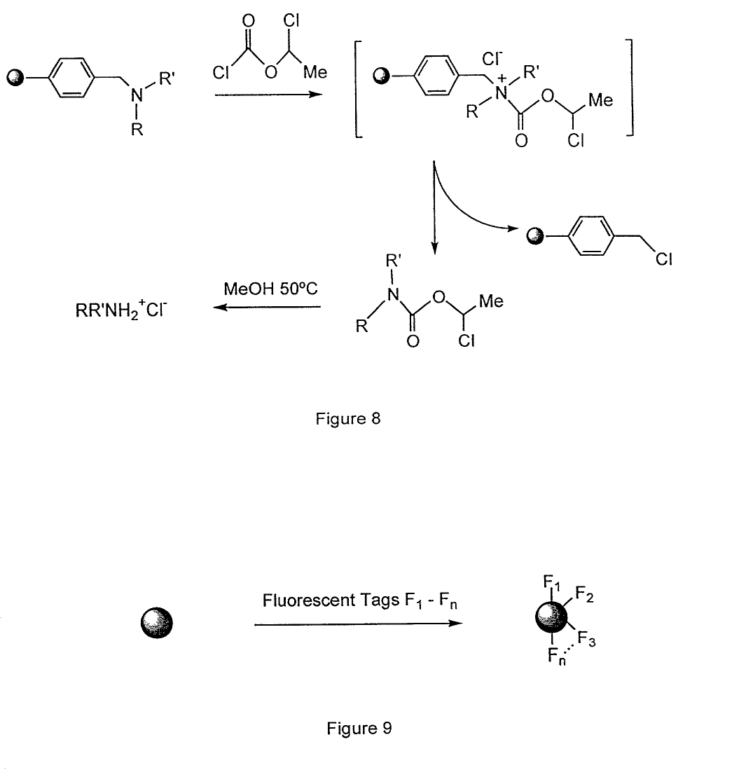

Functionalized polystyrene can be encoded in various ways. For example, as shown in FIG. 6, secondary amine tags can be attached directly to a bromomethylpolystyrene support. Note that in FIG. 6, “x” represents the mole fraction of amine covalently attached to the polystyrene support, and “y” represents the mole fraction of unreacted bromomethyl sites. These unreacted bromomethyl sites can be used for further synthesis, such as attachment of an olefin polymerization catalyst as illustrated in FIG. 7. Alternatively, catalysts can be adsorbed onto the encoded polystyrene support. Note, in FIG. 7, “Mes” denotes the mesityl group.

Other chemical tags such as halocarbons can be covalently attached to the polystyrene resin. Covalent attachment can also be accomplished through a variety of linkages including, but not limited to, ester, amide, amine, ether, urea, thiourea, sulfonamide, alkyl, and aryl. Multiple tags can be attached to effect binary or higher encoding. Binary encoding can also be achieved using an approach similar to that shown in FIG. 4.

Once encoded, the solid supported organometallic compounds can be pooled into a single reactor and screened, for example, for olefin polymerization activity. The group of immobilized catalysts exhibiting the requisite performance can then be decoded to reveal the identity of the catalyst, or the nature of the solid support material.

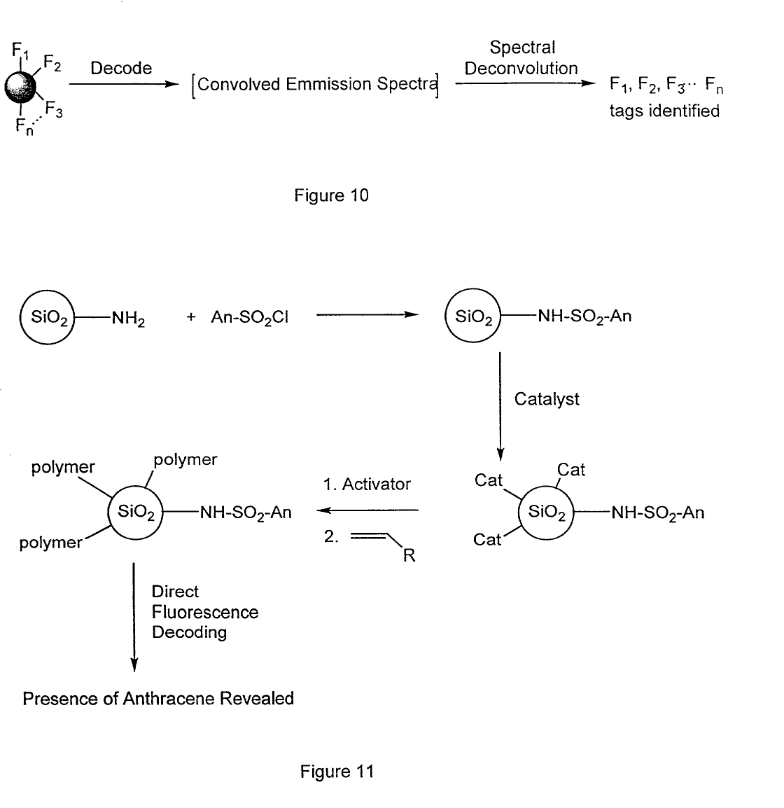

Decoding of secondary amine tags from reaction products can be accomplished through the reaction scheme provided in FIG. 8. The hydrochloride salts of the secondary amines produced from the cleavage routine outlined in FIG. 8 can be neutralized using a base such as Li₂CO₃, derivatized with a fluorescent reagent, such as dansyl chloride, then separated and detected using HPLC coupled with fluorescence detection. Alternatively, the secondary amines can be separated and detected using LC-MS. When halocarbons are used as chemical tags, they can be separated and detected using suitable techniques such as (without limitation) GC-ECD, GC-MS, or LC-MS.

3. Fluorescence Tagging

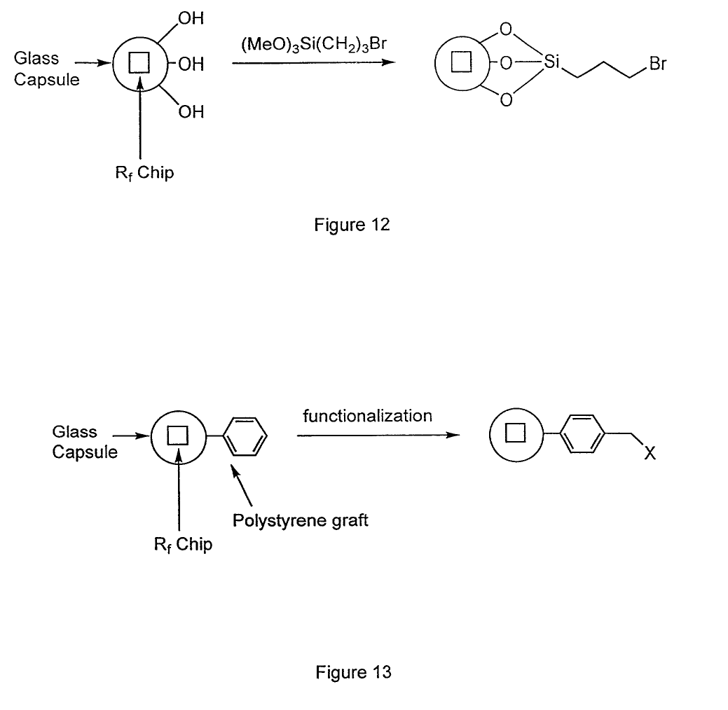

Fluorescence tagging encodes information in the emission spectra of organic or inorganic molecules. Encoding is accomplished through covalent attachment of a fluorescent molecule to a solid support material as shown in FIG. 9. Multiple fluorescent molecules can be attached to effect binary or higher encoding.

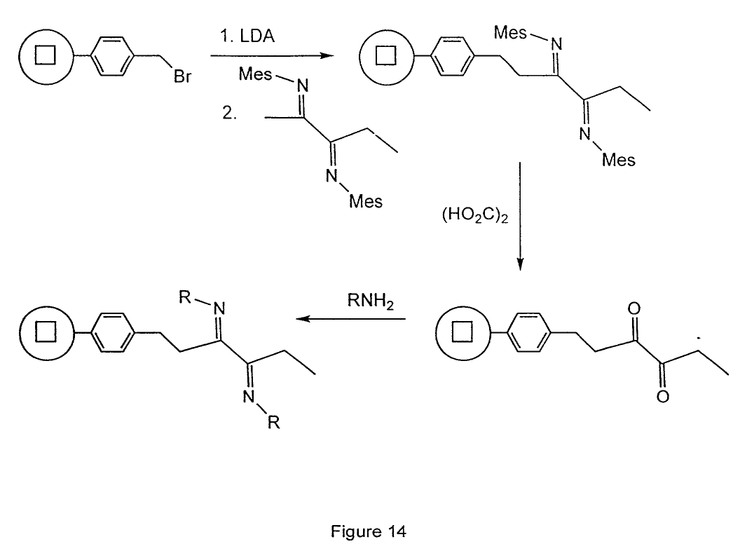

Fluorescence tagging has been applied to crosslinked polystyrene and other resins using both single and multiple tags per resin bead (see, for example, Bradley et al., J. Chem. Soc. Chem. Comm. 735 (1997); Balasubramanian et al., 7(12) Bioorg. Med. Chem. Lett. 1567 (1997)). The encoded information is then read directly from the solid particle through measurement of the convolved emission spectra at several excitation wavelengths, followed by spectral deconvolution to identify the component fluorescent molecules (FIG. 10).

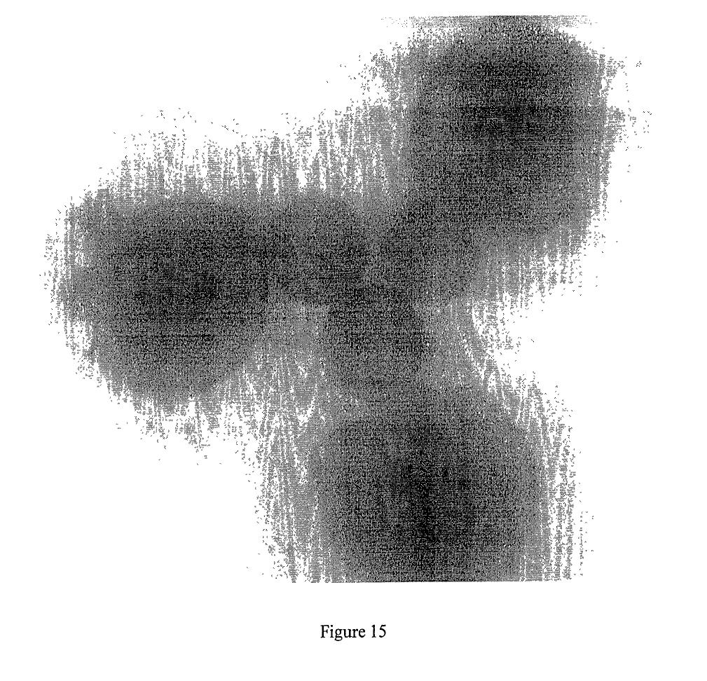

Covalent attachment can be accomplished through a variety of means including, but not limited to ester, amide, amine, ether, urea, thiourea, sulfonamide, alkyl, and aryl linkages. Fluorescent molecules can be conveniently attached to a silica support as illustrated in FIG. 11. The silica support can be derivatized with a suitable functionality, which can be covalently bonded to a molecule such as anthracene (depicted as “An” in FIG. 11) through a suitable linkage. An organometallic compound, such as an olefin polymerization catalyst, can then be adsorbed, covalently attached, or synthesized on the encoded silica. Catalyst activation followed by exposure to an olefin monomer leads to the formation of a polymeric material having an embedded, encoded silica support. Direct fluorescence measurements of the polymeric material allows identification of the chemical tag, thereby revealing the identity of the catalyst.

Alternatively, different silica supports can be tagged with fluorescent molecules and screened for suitability as supports for olefin polymerization catalysts.

4. Radiofrequency Encoding

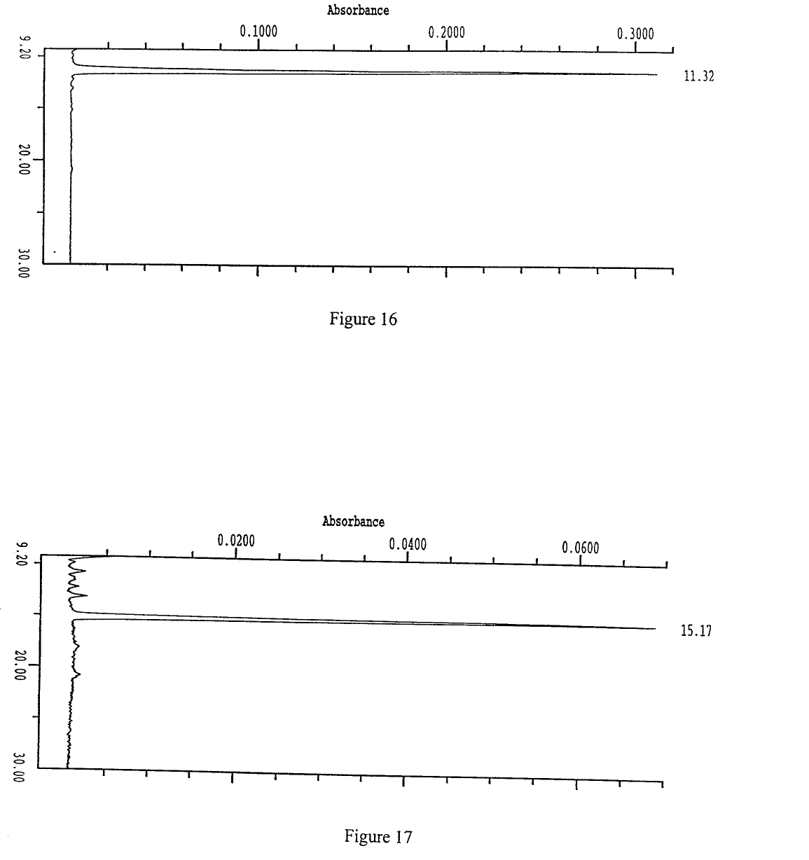

The use of radiofrequency, R_{f}, tags for combinatorial synthesis has recently been described (see, for example, 34 Angew. Chem. Int. Ed. Engl. 2289 (1995); 117 J. Am. Chem. Soc., 10787(1995)). The use of R_{f} tags, which can be encoded by high frequency signals during the synthesis and screening of organometallic libraries, would relieve the burden of post-cleavage characterization of a chemical tag sequence. In addition, the use of a radiofrequency tag, which is a microchip encased in a protective capsule, would allow surface modification of the tag to covalently bind organometallic compounds/inorganic complexes. For example, a silane coupling agent can be attached to the surface of a glass capsule (FIG. 12).

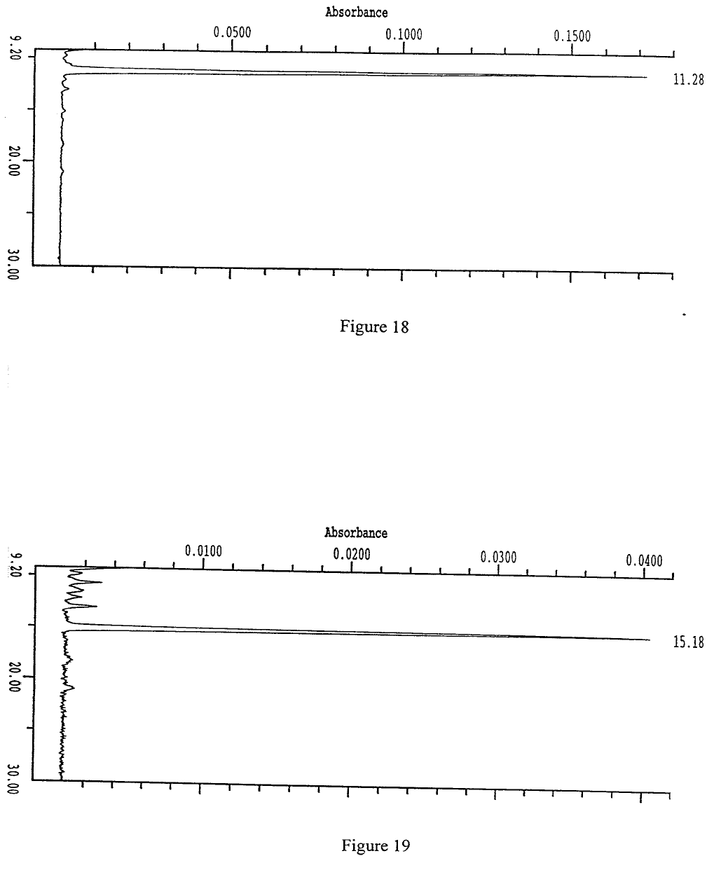

Alternatively, the capsule can be made from a polymeric material or crosslinked polystyrene can be grafted onto the surface of glass capsules, providing a chemically modified surface that can be functionalized using known procedures. For example, in FIG. 13, a polystyrene graft has been functionalized with a halogen, represented by the symbol “X.” The procedure illustrated in FIG. 13 can produce a modified surface with a higher loading capacity than the surface of a glass capsule alone.

The functionalized R_{f} tags illustrated in FIG. 12 and 13 can be used for further synthesis, such as the preparation of member compounds of an organometallic library/inorganic complex library. For instance, the functionalized glass capsule illustrated in FIG. 13 can be used to prepare encoded bis-imine ligands as shown in Scheme 14. Transition metals can then be attached to the library compounds to form encoded bis-imine complexes.

Thus, libraries of organometallic compounds/inorganic complexes can be prepared that are covalently attached to the surface of a glass encapsulated R_{f} chip. Such libraries can be pooled and screened for a desired property, such as catalytic activity. The high frequency signals encoded in the R_{f} chips can then be read to identify library member compounds have desired properties.

5. Encoding By Nuclear Transmutation and Nuclear Excitation

The invention also includes encoding the details of a multi-step synthetic route in the mass fractions of radioactive nuclei or their stable daughter nuclei contained within the bead or other solid support. Supports can also be encoded and used in a parallel or rapid serial synthesis of a combinatorial library of organometallic compounds/inorganic complexes.

Starting with a solid support of a known nuclear composition of several selected “parent” isotopes, each synthetic step is encoded using exposure to one or many of several types of radiation (neutrons, high energy photons, protons, electrons) at selected energies for selected exposures to transmute the isotopic composition. The exposure will be unique to a specific synthetic step. For example, the addition of a phenol group in one step may involve irradiation for 2 minutes with neutrons, while the addition of an ethanol group would involve a 4 minute irradiation. Thus, the isotopic abundance of the new isotopes would be twice as high in the ethanol added beads than in the phenol added beads.

The nuclear transmutation may involve, for example, the absorption of a neutron by the nucleus to form a new isotope increased in mass by one mass unit. The subsequent radioactive decay of the new isotope may then reduce the ratio of the new isotope to the parent isotope in time at a known rate. In one embodiment, the number of different parent isotopes in the original solid support must equal the number of steps to encode. Using the time of each irradiation and the time at which the final analysis of the bead is performed the specific code can be read.

In another embodiment, a compound meeting some predefined screening criteria or a “hit” is decoded by placing the bead within a mass spectrometer and determining the isotopic abundance of the remaining parent isotopes and the new transmuted isotopes. One can determine the specific code or identity of the compound from the isotopic ratios. In still another embodiment, the rate of radioactive decay is also measured and used with the isotopic ratios to decode the synthetic steps.

To implement encoding and decoding by nuclear transmutation and excitation requires a radiation source, automated library handling devices and a shielded beam path or irradiation, automated radiation counting and mass spectrometry.

### EXAMPLES

The following examples represent a variety of embodiments of the present invention.

### Example 1

Example one details the attachment (e.g., encoding) of secondary amines to a polystyrene support (see FIG. 6), and the preparation of a tagged polystyrene supported bis-imine nickel and palladium olefin polymerization catalysts.

### Example 1.1

### Attachment of Methylpentylamine

Bromomethylpolystyrene (1 g, 0.51 mmol, 0.51 mmol/g) and methylpentylamine (19 μL, 0.15 mmol) were combined in 10 mL N-methylpyrolidine and heated to 50° C. for 20 hours after which the resin was isolated by filtration and washed with N-methylpyrolidine, 4:1 THF/saturated Li₂CO₃ in H₂O (2×20 mL), 4:1 THF/H₂O (2×20 mL), THF (2×20 mL), CH₂Cl₂ (2×20 mL), and Et₂O (2×20 mL). The resultant resin (1 g) was dried under vacuum.

### Example 1.2

### Attachment of Dipentylamine

Bromomethylpolystyrene (1 g, 0.51 mmol, 0.51 mmol/g) and methylpentylamine (31 μL, 0.15 mmol) were combined in 10 mL N-methylpyrolidine and heated to 50° C. for 20 hours after which the resin was isolated by filtration and washed with N-methylpyrolidine, 4:1 THF/saturated Li₂CO₃ in H₂O (2×20 ml), 4:1 THF/H₂O (2×20 mL), THF (2×20 ml), CH₂Cl₂ (2×20 ml), and Et₂O (2×20 mL). The resultant resin (1 g) was dried under vacuum.

### Example 1.3

### Alkylation of (2,4,6-Me₃C₆H₂)₂(DAB)MeEt with Tagged Bromomethylpolystyrene

Both of the products from Examples 1.1 and 1.2 were treated in the following manner. To a cooled solution (0° C.) of (2,4,6-Me₃C₆H₂)₂DAB(Me)Et (0.17 g 0.5 mmol, DAB≡diazabutadiene) in 10 mL of dry THF under nitrogen was added LDA (330 μL, 0.5 mmol, 1.5 M in THF). The resultant mixture was stirred at 0° C. for 45 minutes whereupon 0.5 g tagged bromomethylpolystyrene was added. The resulting suspension was stirred for 90 minutes at ice temperature and 17 hours at room temperature. Each resin was isolated by filtration and washed with THF (2×25 ml), H₂O (2×25 ml), THF (2×25 ml), CH₂Cl₂ (2×25 ml), and then dried under vacuum to yield 0.5 g of the desired bright yellow resins.

### Example 1.4

### Preparation of Tagged [(2,4,6-Me₃C₆H₂)₂DAB(Me)Et]NiBr₂ Polystyrene

Both resins prepared from Example 1.3 were treated in the following manner. 0.40 g of resin and 0.16 g (DME)NiBr₂ (0.53 mmol, DME=dimethoxyethane) were suspended in 40 mL of dry CH₂Cl₂ under nitrogen and stirred at room temperature for 24 hours. The resin was then washed with CH₂Cl₂ (2×30 mL), and dry acetone (2×30 mL), to give 0.40 g of encoded [(2,4,6-Me₃C₆H₂)₂DAB(Me)Et]NiBr₂ polystyrenes as a red-brown resins. The loading capacities of the resins were:

Methylpentylamine tagged resin: 0.37 mmol/g (based upon nickel analysis)

Dipentylamine tagged resin: 0.31 mmol/g (based upon nickel analysis)

### Example 1.5

### Preparation of Tagged [(2,4,6-Me₃C₆H₂)₂DAB(Me)Et]PdMeCl Polystyrene.

To a methylpentylamine tagged polystyrene alkylated with (2,4,6-Me₃C₆H₂)₂(DAB)MeEt prepared in a manner similar to that described in Example 1.3 (0.4 g) was added 0.4 g of (1,5-cyclooctadiene)PdMeCl in 10 mL of CH₂Cl₂. The resultant mixture was stirred for 2 hours whereupon the resultant red/brown resin was collected by filtration and washed with CH₂Cl₂ (2×30 mL), toluene (2×30 mL) and pentane (2×30 mL). The loading of the resin was determined to be 0.32 mmol/g (based on palladium analysis).

### Example 2

Example 2 illustrates the utility of the encoded organometallic compounds as olefin polymerization catalysts.

### Example 2.1

### Polymerization of Ethylene Using Methylpentylamine Tagged [(2,4,6-Me₃C₆H₂)₂DAB(Me)Et]NiBr₂ Polystyrene

0.20 g of methylpentylamine tagged [(2,4,6-MeC₆H₂)₂DAB(Me)Et]NiBr₂ polystyrene was suspended in 15 mL toluene within a high pressure reaction vessel. A solution of methylalumoxane was added which caused the color of the resin beads to change from red-brown to violet blue (3 mL 10 wt % in toluene). The mixture was stirred for 1 minute whereupon an overpressure of ethylene gas was introduced into the reaction vessel (20 psi). After stirring for 1 hour the reaction was quenched with MeOH/H₂O and the pH reduced to <1 with HCl to remove the excess methylalumoxane. The product from the organic layer was the collected by filtration and washed with H₂O and acetone to yield 3.8 g of polyethylene granules.

### Example 2.2

### Polymerization of Ethylene Using a Pooled Mixture of Methylpentylamine Tagged (2,4,6-Me₃C₆H₂)₂DAB(Me)Et]NiBr₂ Polystyrene and Dipentylamine Tagged [(2,4,6-Me₃C₆H₂)₂DAB(Me)Et]NiBr₂ Polystyrene

A mixture of 0.015 g of methylpentylamine tagged [(2,4,6-Me₃C₆H₂)₂DAB(Me)Et]NiBr₂ polystyrene and 0.015 g of dipentylamine tagged [(2,4,6-Me₃C₆H₂)₂DAB(Me)Et]NiBr₂ polystyrene was suspended in 5 mL toluene within a high pressure reaction vessel. A solution of methylalumoxane was added which caused the color of the resin beads to change from red-brown to violet blue (0.5 mL 10 wt % in toluene). The mixture was stirred for 1 minute whereupon an overpressure of ethylene gas was introduced into the reaction vessel (20 psi). After stirring for 1 hour the reaction was quenched with MeOH/H₂O and the pH reduced to <1 with HCl to remove the excess methylalumoxane. The product from the organic layer was the collected by filtration and washed with H₂O and acetone to yield 1.5 g of polyethylene granules.

### Example 2.3

### Polymerization of Ethylene Using a Pooled Mixture of Methylpentylamine Tagged (2,4,6-Me₃C₆H₂)₂DAB(Me)Et]PdMeCl Polystyrene and Dipentylamine Tagged [(2,4,6-Me₃C₆H₂)₂DAB(Me)Et]NiBr₂ Polystyrene

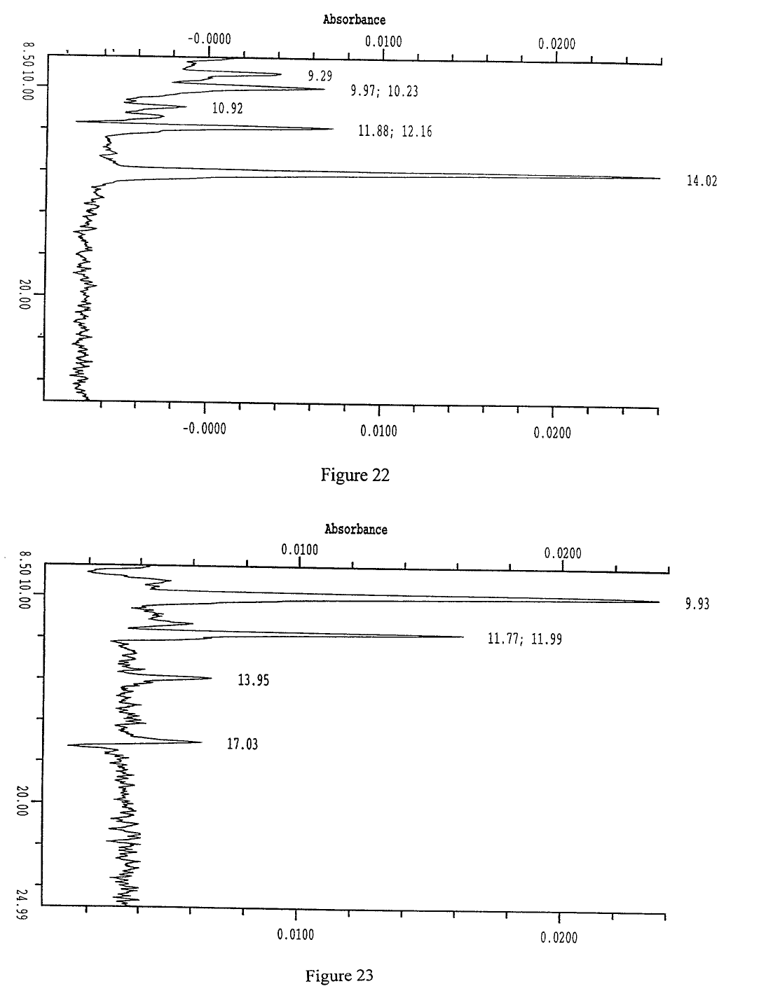

A mixture of 0.1 g of methylpentylamine tagged [(2,4,6-Me₃C₆H₂)₂DAB(Me)Et]PdMeCl polystyrene and 0.1 g of dipentylamine tagged [(2,4,6-Me₃C₆H₂)₂DAB(Me)Et]NiBr₂ polystyrene was suspended in 30 mL toluene within a thick walled glass ampule. A solution of methylalumoxane (0.5 mL 10 wt % in toluene) was added which caused the color of the resin beads to darken significantly. The mixture was stirred for 1 minute whereupon an overpressure of ethylene gas was introduced into the reaction vessel (20 psi). After stirring for 6 hours the reaction was quenched with MeOH/H₂O and the pH reduced to <1 with HCl to remove the excess methylalumoxane. The product from the organic layer was the collected by filtration and washed with H₂O and acetone to yield 0.52 g of polyethylene granules. An enlarged image of a representative sample of the product from this pooled reaction is shown in FIG. 15. The image reveals the presence of two types of polyethylene granules, easily distinguishable by size. Large and small granules were selected for decoding (see Example 3).

### Example 3

Example 3 illustrates the decoding of the amine tags from the encoded polystyrene resins and encoded polymer granules and separation and detection of the derivatized amines using HPLC with fluorescence detection. These examples demonstrate the utility of the technique in distinguishing the performances of different catalysts in a pooled polymerization reaction.

### Example 3.1

### Cleavage of Amine Tags

The following samples were subjected to the cleavage procedure:

Sample 1: Methylpentylamine encoded bromomethyl polystyrene. Product from Example 1.1. 10-20 resin beads were selected.

Sample 2: Dipentylamine encoded bromomethyl polystyrene. Product from Example 1.2. 10-20 resin beads were selected.

Sample 3: Methylpentylamine encoded [(2,4,6-Me₃C₆H₂)₂DAB(Me)Et]NiBr₂ polystyrene. Product from Example 1.4. 10-20 resin beads were selected.

Sample 4: Dipentylamine encoded [(2,4,6-Me₃C₆H₂)₂DAB(Me)Et]NiBr₂ polystyrene. Product from Example 1.4. 10-20 resin beads were selected.

Sample 5: 20-30 granules of encoded polyethylene product from Example 2.2.

Sample 6: 1 granule of encoded polyethylene product from Example 2.1.

Sample 7: 1 large granule of encoded polyethylene product from Example 2.3 (see FIG. 15).

Sample 8: 1 small granule of encoded polyethylene product from Example 2.3 (see FIG. 15).

In a typical decoding experiment 100 μL of α-chloroethylchloroformate and 150 μL methylene chloride were added to the sample. The resultant suspensions were agitated for 5 hours at room temperature whereupon each sample was evaporated to dryness. To each sample was then added 300 μL MeOH and the resultant suspensions were heated at 50° C. overnight after which time the samples were evaporated to dryness. These samples were submitted for HPLC as described in Example 3.2.

### Example 3.2

### Separation and Detection of Amine Tags by HPLC.

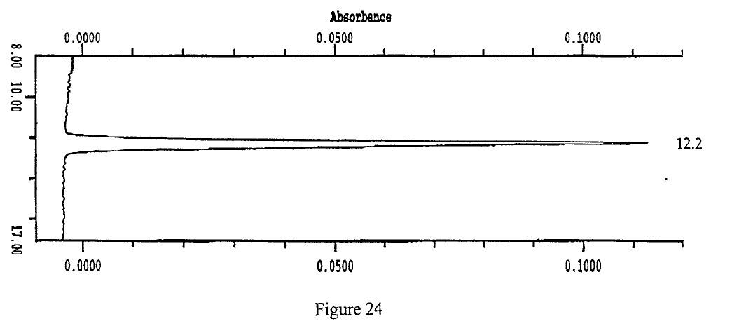

In a typical experiment 40 μL saturated Li₂CO₃ in H₂O, 80 μL dansyl chloride and 880 μL of 2:1 acetonitrile:H₂O were added to each sample of Example 3.1. Dansyl chloride forms complexes with amines,which can be detected by fluorescence detection. The excitation frequency was 352 nm and the detection frequency was 510 nm. The separation of amines was performed on a C₁₈ column using a 2:1 acetonitrile: H₂O mobile phase. The injection volume was 20 μL.HPLC traces obtained from of Samples 1-8 in Example 3.1 are provided in FIGS. 16-23, respectively. Retention times of the dansyl chloride methylpentylamine complex are 11.3 minutes (Samples 1-6) and 10 minutes (Samples 7 and 8); . Retention times of the dansyl chloride dilpentylamine complex are 15.2 minutes (Samples 1-6) and 14 minutes (Samples 7 and 8). Differences in retention times between two sets of samples are the result of optimizing HPLC parameters prior to decoding Sample 7 and 8.

### Example 4

Example 4 details the attachment (e.g., encoding) of secondary amines to a silica support (see FIG. 2), and the adsorption of olefin polymerization catalysts onto the tagged silica.

### Example 4.1

### Preparation of Dipentylaminomethyl(phenylethyl)trimethoxysilane

A solution containing 0.511 g (1.86 mmol) ((chloromethyl)phenylethyl)trimethoxysilane in 7.5 mL of THF was treated with 836 μL (3.72 mmol) of dipentylamine. The resultant mixture was heated to 85-90° C. for 20 hours, whereupon it was cooled to room temperature and filtered. Solvent was removed from the filtrate under reduced pressure and the resultant crude product was vacuum distilled to produce Dipentylaminomethyl(phenylethyl)trimethoxysilane as an oily solid (484 mg, 66% yield).

### Example 4.2

### Preparation of Methylpentylaminomethyl(phenylethyl)trimethoxysilane

The preparation of Methylpentylaminomethyl(phenylethyl)trimethoxysilane was performed in a manner similar to that described in 4.1

### Example 4.3

### Preparation of Silica Tagged (1%) With Dipentylamine

A sample of 245 mg of silica (3.0 mmol[-OH]/g, 0.74 mmol) was treated with a solution of 2.9 mg (7.4 μmmol, 1% loading) dipentylaminomethyl(phenylethyl)trimethoxysilane in 5 mL toluene. The resultant suspension was heated to reflux for 1 hour whereupon the silica was isolated by filtration, washed with 2×5 mL hexane and dried under vacuum.

### Example 4.4

### Adsorption of an Olefin Polymerization Catalyst onto a 1% Tagged Silica Support

To a stirred slurry of a sample of 100 mg of silica tagged with dipentylamine, (prepared as described in Example 4.3) in 5 mL of toluene, was added 0.7 mL of methylalumoxane (10 wt. % solution in toluene), and 4.3 mL of toluene over a period of 10 minutes. After additional stirring for 30 minutes, the resultant mixture was heated to 75° C. and stirred for 4 hours. To the resultant mixture was added 10 mL of a solution containing 0.05 g of rac-[(Me₂Si(C₉H₆)₂]ZrCl₂ in 100 mL toluene. This mixture was stirred at 75° C. for a further 2 hours whereupon the solvent was removed to produce a free flowing powder.

### Example 5

### Cleavage of Amine Tags

Example 5 details the decoding silica tagged with dipentylamine. The tagged silica sample prepared in Examples 4.3 was treated as follows. 1 mg of tagged silica was suspended in 250 μL of CH₂Cl₂ along with 5 μL of α-chloroethylchloroformate. The mixture was stirred overnight whereupon the silica was removed by filtration. The solvent from the filtrate was evaporated and the residue was dissolved in methanol and heated to reflux for 3 hours. The methanol was then removed under reduced pressure and the sample was identified by HPLC. This sample was treated as described in Example 3.2 except that prior to injection the sample was diluted by a factor of 1000 with 2:1 acetonitrile:water. The retention time for derivatized dipentylamine was 12.2 minutes, as deduced by comparison with calibration standards. The HPLC trace for this sample is shown in FIG. 24.

### Example 6

Example 6 illustrates the encoding and decoding of fluorescent tags on a silica support.

### Example 6.1

### Preparation of 10% Loaded Aminopropyl Silica (10% AP-Silica)

To 10 g of silica (90μ, 300 m²/g, 1.2 mmol OH/g; dried at 150° C. for 24 h) suspended in 50 mL of anhydrous toluene was added 267 mg (1 mmol) of 3-aminopropyltriethoxysilane and the slurry was heated with stirring under N₂ for 1 h. The flask was fitted with a distillation head and the volatiles were distilled off to near dryness. The silica was collected on a fritted glass funnel and washed with 2×50 mL of toluene, 3×50 mL THF, then dried in vacuo for 12 hours.

### Example 6.2

### Preparation of 1% Tagged Silica

10% AP-Silica was tagged with two fluorescent tags: Anthracene-SO₂Cl and Pyrene-SO₂Cl (both denoted generically as tag-SO₂Cl) in the following manner. To a slurry of 1.0 g of 10% AP-Silica (0.12 mmol NH₂/g) in 20 mL of anhydrous CH₂Cl₂ was added 0.012 mmol Tag-SO₂Cl and the mixture was stirred for 12 h under N₂. The tagged silica was filtered under N₂, washed with 2×10 mL CH₂Cl₂ and 3×10 mL THF, then dried in vacuo for 12 hours.

### Example 6.3

### Preparation of Doubly 1% Tagged Silica

To a slurry of 1% tagged silica in 20 mL of anhydrous CH₂Cl₂ was added 0.012 mmol tag-SO₂Cl (where tag-SO₂Cl is a different tag to the tag attached to 1% tagged silica) and the mixture was stirred for 12 hours under N₂. The silica was collected, washed and dried as described in Example 6.2.

### Example 6.4

### Treatment of Silica With Methylalumoxane

To 100 mg of tagged silica suspended in 1 mL of anhydrous toluene was added 5 mL of 25% w/w of methylalumoxane in toluene. The mixture was heated to 60° C. under N₂ for 1 hour. The silica was then filtered and washed with 3×5 ml toluene followed by 3×5 mL anhydrous pentane. The silica was then dried in vacuo for 1 hour.

### Example 6.5

### Fluorescence Measurement of Loaded Silicas

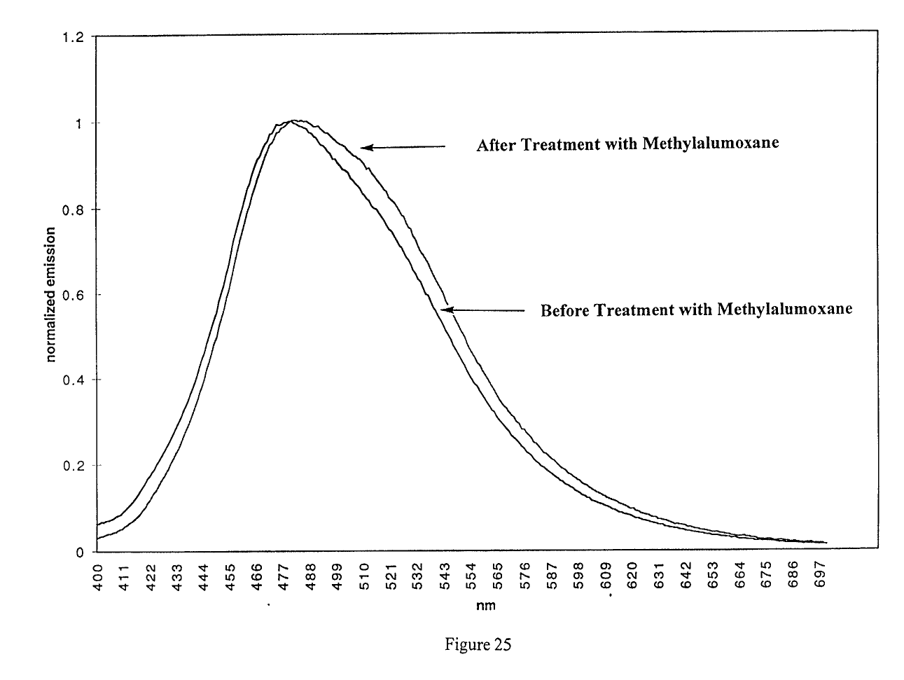

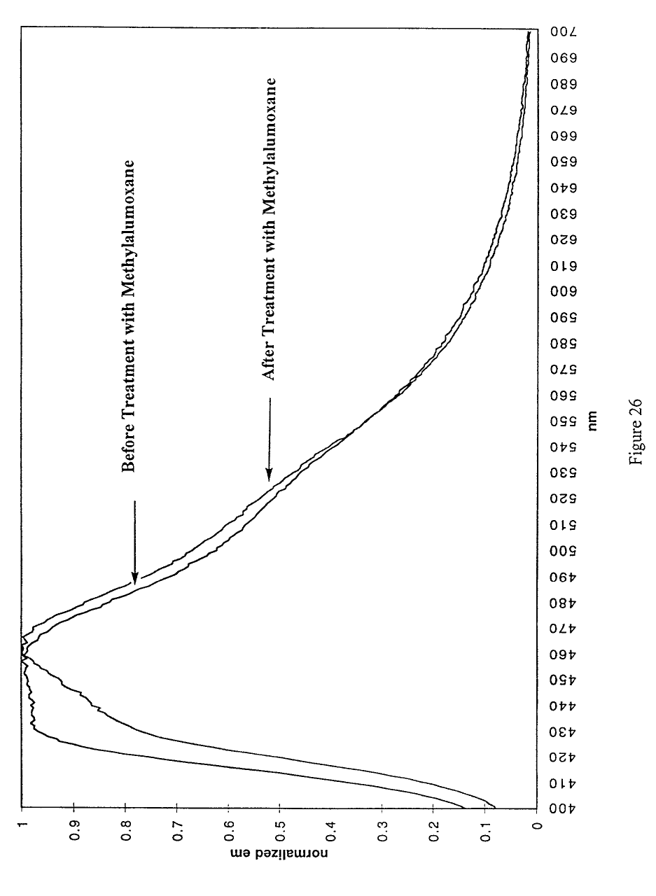

Fluorescence measurements were performed directly on the silica samples prepared in Example 6.2 and Example 6.4 on a SPEX Fluorolog Spectrophotometer. Samples were mounted vertically in a glass-faced A1 block positioned at 45 between source and detector. Emission scans were collected between 360-800 nm at several excitation wavelengths between 350-400 nm. FIG. 25 presents the emmision spectra of the pyrene tagged silica both before and after treatment with methylalumoxane and FIG. 26 represents the emmision spectra of the anthracene tagged silica both before and after treatment with methylalumoxane.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated herein by reference for all purposes.

We claim:

1. A method of identifying member compounds of a combinatorial library, the combinatorial library having a total number of member compounds equal to N, each of the member compounds immobilized on separate solid supports, and the separate solid supports comprising a total number of distinct compositions equal to M, the method comprising the steps of:
immobilizing each of the member compounds on separate solid supports so as to form immobilized member compounds of the formula AᵢBⱼ, wherein A represents the separate solid supports, B represents the member compounds, subscript i refers to a particular solid support composition and is any positive integer less than or equal to M, and subscript j refers to a particular member compound and is any positive integer less than or equal to N;
encoding the immobilized member compounds by attaching at least one tag to each of the immobilized member compounds so as to form tagged immobilized member compounds, wherein the at least one tag is detectable and unique for each AᵢBⱼ;
combining the tagged immobilized compounds to allow screening of the tagged immobilized compounds;
selecting at least a subset of tagged immobilized compounds based on a screening criterion; and
decoding the subset of tagged immobilized compounds by detecting the at least one tag of each AᵢBⱼ belonging to the subset of tagged immobilized compounds so as to identify member compounds and solid supports meeting the screening criterion;
wherein the member compounds are selected from the group consisting of organometallic compounds, inorganic complexes, and mixtures thereof.

2. The method of claim 1, wherein the immobilizing step further comprises the step of:
synthesizing each of the member compounds on separate solid supports by sequential addition of reagents.

3. The method of claim 2, wherein the encoding step is carried out concurrently with the synthesizing step.

4. The method of claim 2, wherein the encoding step further comprises the step of attaching a set of tags to each of the immobilized member compounds, wherein the set of tags is unique for each sequential addition of reagents.

5. The method of claim 1, wherein the immobilizing step further comprises the step of:
covalently linking each of the member compounds to the separate solid supports.

6. The method of claim 1, wherein the immobilizing step further comprises the step of:
adsorbing each of the member compounds on the separate solid supports.

7. The method of claim 1, wherein the encoding step is carried out before the immobilizing step.

8. The method of claim 1, wherein more than one copy of the particular member compound is immobilized on each AᵢBⱼ.

9. The method of claim 1, wherein more than one copy of each AᵢBⱼ is formed in the immobilizing step.

10. The method of claim 1, wherein the solid supports are selected from the group consisting of organic polymers, inorganic solids, and mixtures thereof.

11. The method of claim 10, wherein the solid supports are selected from the group consisting of polystyrene, polysiloxane, polyethylene glycol, polypropylene glycol, polytetrafluoroethyelene, silica, alumina, aluminosilicate, magnesium chloride, and mixtures thereof.

12. The method of claim 1, wherein the at least one tag is a molecule, atom or ion attached to the solid supports.

13. The method of claim 12, wherein the at least one tag is adsorbed on the solid supports.

14. The method of claim 12, wherein the at least one tag is covalently linked to the solid supports.

15. The method of claim 14, wherein the at least one tag is attached to the solid support using a linker compound.

16. The method of claim 1, wherein the at least one tag is selected from the group consisting of amine, halocarbon, and mixtures thereof.

17. The method of claim 16, wherein the at least one tag is a secondary amine.

18. The method of claim 1, wherein the at least one tag is a fluorescent molecule.

19. The method of claim 1, wherein the at least one tag is a radioisotope, the method further comprising the steps of:
exposing the at least one tag to radiation resulting in a transmuted radioisotope; and
decoding the tag by detecting the transmuted radioisotope.

20. The method of claim 1, wherein the at least one tag is a radiofrequency tag comprising a microchip encased in a capsule, the capsule having a surface that can be used to immobilize member compounds.

21. The method of claim 20, wherein the capsule surface is glass.

22. The method of claim 21, further comprising the step of treating the capsule surface with a silane coupling agent so as to allow member compounds to be covalently attached to the capsule surface.

23. The method of claim 21, further comprising the steps of:
grafting a polymer on the capsule surface; and
functionalizing the polymer so as to provide binding sites for the member compounds.

24. The method of claim 1, wherein the at least one tag is a molecule, atom or ion attached to the member compounds.

25. The method of claim 1, wherein the at least one tag is a molecule, atom or ion attached to a reaction product of the member compounds.

26. The method of claim 1, wherein the screening criterion of the selecting step is either a catalytic activity of a member compound or a polymer characteristic of a polymer made using a member compound.

27. The method of claim 1, wherein the decoding step further comprises the step of cleaving the at least one tag of each AᵢBⱼ belonging to the subset of tagged immobilized compounds meeting the screening criterion.

28. The tagged immobilized compounds made in accordance with the method of claim 1.

29. A method of screening member compounds of a combinatorial library for catalytic activity, the combinatorial library having a total number of member compounds equal to N, each of the member compounds immobilized on separate solid supports, and the separate solid supports comprising a total number of distinct compositions equal to M, the method comprising the steps of:
immobilizing each of the member compounds on separate solid supports so as to form immobilized member compounds of the formula AᵢBⱼ, wherein A represents the separate solid supports, B represents the member compounds, subscript i refers to a particular solid support composition and is any positive integer less than or equal to M, and subscript j refers to a particular member compound and is any positive integer less than or equal to N;
encoding the immobilized member compounds by attaching at least one tag to each of the immobilized member compounds so as to form tagged immobilized member compounds, wherein the at least one tag is detectable and unique for each AᵢBⱼ;
combining the tagged immobilized compounds to form a mixture of tagged immobilized compounds;
contacting the mixture of tagged immobilized compounds with a reactant;
selecting at least a subset of tagged immobilized compounds meeting a predefined measure of catalytic activity; and
decoding the subset of tagged immobilized compounds by detecting the at least one tag of each AᵢBⱼ belonging to the subset of tagged immobilized compounds so as to identify member compounds and solid supports meeting the predefined measure of catalytic activity;
wherein the member compounds are selected from the group consisting of organometallic compounds, inorganic complexes, and mixtures thereof.

30. The method of claim 29, wherein the reactant is a nonmetal.

31. The method of claim 29, wherein the reactant is an olefin, functionalized olefin, diolefin or acetylenically unsaturated compound.

32. The method of claim 31, wherein the reactant is selected from the group consisting of ethylene, propylene, 1-butene, isobutylene, 4-methyl-1-pentene, 1-octene, 1-hexene, isoprene, styrene, a diene, olefinic esters, olefinic carboxylic acids, olefinic nitrites and mixtures thereof.

33. The method of claim 29, wherein the contacting step results in reaction products having the formula Cₖₗ, wherein C represents the reaction products, subscript k is any positive integer less than or equal to M, and subscript l is any positive integer less than or equal to N, so that each C_{k,l} represents a particular reaction product catalyzed by AᵢBⱼ when k equals i and l equals j.

34. The method of claim 33, wherein the reaction products adhere to the solid supports forming bound reaction products having the formula C_{k,l}[AᵢBⱼ] in which k equals i and l equals j.

35. The method of claim 34, wherein the bound reaction products are polymers.

36. The method of claim 35, wherein the polymers are polyolefins.

37. The tagged immobilized compounds made in accordance with the method of claim 29.

38. An encoded combinatorial library comprising:
member compounds immobilized on separate solid supports having the formula AᵢBⱼ, wherein the total number of member compounds equals N and the total number of solid support compositions equals M, A represents the separate solid supports, B represents the member compounds, subscript i refers to a particular solid support composition and is any positive integer less than or equal to M, and subscript j refers to a particular member compound and is any positive integer less than or equal to N, wherein the member compounds are selected from the group consisting of organometallic compounds, inorganic complexes, and mixtures thereof;
at least one tag, wherein the at least one tag is attached to each of the immobilized member compounds, the at least one tag is detectable and unique for each AᵢBⱼ so that when the immobilized member compounds are combined with a reactant, each AᵢBⱼ exhibiting a predefined measure of catalytic performance can be identified by detecting the at least one tag that is unique to each AᵢBⱼ.

39. The encoded combinatorial library of claim 38, wherein each of the member compounds are synthesized on the separate solid supports by sequential addition of reagents.

40. The encoded combinatorial library of claim 39, wherein the at least one tag further comprises a set of tags, the set of tags attached to each of the immobilized member compounds, wherein the set of tags is unique for each sequential addition of reagents.

41. The encoded combinatorial library of claim 38, wherein each of the member compounds are covalently linked to the separate solid supports.

42. The encoded combinatorial library of claim 38, wherein each of the member compounds are adsorbed on the separate solid supports.

43. The encoded combinatorial library of claim 38, wherein more than one copy of the particular member compound is immobilized on each AᵢBⱼ.

44. The encoded combinatorial library of claim 38, further comprising at least one copy of each AᵢBⱼ.

45. The encoded combinatorial library of claim 38, wherein the solid supports are selected from the group consisting of organic polymers, inorganic solids, and mixtures thereof.

46. The encoded combinatorial library of claim 45, wherein the solid supports are selected from the group consisting of polystyrene, functionalized polystyrene, polysiloxane, polyethylene glycol, polypropylene glycol, polytetrafluoroethyelene, silica, alumina, aluminosilicate, functionalized alumina, functionalized silica, magnesium chloride, and mixtures thereof.

47. The encoded combinatorial library of claim 38, wherein the at least one tag is a molecule, atom or ion attached to the solid supports.

48. The encoded combinatorial library of claim 47, wherein the at least one tag is adsorbed on the solid supports.

49. The encoded combinatorial library of claim 47, wherein the at least one tag is covalently linked to the solid supports.

50. The encoded combinatorial library of claim 49, wherein the at least one tag is attached to the solid support using a linker compound.

51. The encoded combinatorial library of claim 38, wherein the at least one tag is selected from the group consisting of amine, halocarbon, and mixtures thereof.

52. The encoded combinatorial library of claim 51, wherein the at least one tag is a secondary amine.

53. The encoded combinatorial library of claim 38, wherein the at least one tag is a fluorescent molecule.

54. The encoded combinatorial library of claim 38, wherein the at least one tag is a radioisotope.

55. The encoded combinatorial library of claim 38, wherein the at least one tag is a radiofrequency tag comprising a microchip encased in a capsule, the capsule having a surface that can be used to immobilize member compounds.

56. The encoded combinatorial library of claim 55, wherein the capsule surface is glass.

57. The encoded combinatorial library of claim 56, wherein the capsule surface is treated with a silane coupling agent so as to allow member compounds to be covalently attached to the capsule surface.

58. The encoded combinatorial library of claim 56, wherein the capsule further comprises:
a layer of polymer grafted on the glass surface;
wherein the polymer is functionalized so as to provide binding sites for the member compounds.

59. The encoded combinatorial library of claim 38, wherein the at least one tag is a molecule attached to the member compounds.

60. The encoded combinatorial library of claim 38, wherein the at least one tag is a molecule attached to a reaction product of the member compounds.

61. The encoded combinatorial library of claim 38, wherein the at least one tag of each AᵢBⱼ is cleavable.
